(19) United States
(12) Patent Application Publication
(10) Pub. No.: US 2022/0054259 A1
(43) Pub. Date: Feb. 24, 2022

(54) Flexible Anchor For Prosthetic Heart Valve

(71) Applicant: Tendyne Holdings, Inc., St. Paul, MN (US)

(72) Inventors: Philip Osterbauer, Wyoming, MN (US); Preston James Huddleston, Maplewood, MN (US); Tracee Eidenschink, Wayzata, MN (US)

(73) Assignee: Tendyne Holdings, Inc., St. Paul, MN (US)

(21) Appl. No.: 17/384,259

(22) Filed: Jul. 23, 2021

Related U.S. Application Data
(60) Provisional application No. 63/067,061, filed on Aug. 18, 2020.

(51) Int. Cl. A61F 2/24

(52) U.S. Cl. CPC A61F 2/2418; A61F 2002/0081; A61F 2220/0016

(57) ABSTRACT

A prosthetic heart valve may include a valve portion, a tether connected to the valve portion, and an anchor for connecting the tether to the wall of the heart. The prosthetic heart valve system includes a collapsible and expandable stent, a plurality of prosthetic leaflets coupled to the stent, a collapsible anchor, and a tether configured to couple the stent to the anchor, wherein the collapsible anchor includes a flexible frame body defining a plurality of collapsible cells, each cell having at least one side shared with a neighboring cell, a center adjoining point defined by the cells formed in the flexible frame body, the center adjoining point configured to receive the tether, and a first tip end and a second tip end defining a major axis of the flexible frame body, the major axis passing through the center adjoining point.

### CROSS-REFERENCE TO RELATED APPLICATIONS

This application claims benefit of United States Provisional Application Ser. No. 63/067,061 filed Aug. 18, 2020, which is incorporated by reference in its entirety.

### BACKGROUND OF THE DISCLOSURE

Valvular heart disease, and specifically aortic and mitral valve disease, is a significant health issue in the United States. Annually, approximately 90,000 valve replacements are performed in the United States. Traditional valve replacement surgery, the orthotopic replacement of a heart valve, is an “open heart” surgical procedure. Briefly, the procedure necessitates a surgical opening of the thorax, initiation of extra-corporeal circulation with a heart-lung machine, stopping and opening the heart, excision and replacement of the diseased valve, and re-starting of the heart. While valve replacement surgery typically carries a 1-4% mortality risk in otherwise healthy persons, a significantly higher morbidity is associated with the procedure, largely due to the necessity for extra-corporeal circulation. Further, open heart surgery is often poorly tolerated in elderly patients. Thus, if the extra-corporeal component of the procedure could be eliminated, morbidities and cost of valve replacement therapies would be significantly reduced.

While replacement of the aortic valve in a transcatheter manner is the subject of intense investigation, lesser attention has been focused on the mitral valve. This is in part reflective of the greater level of complexity associated with the native mitral valve and thus a greater level of difficulty with regard to inserting and anchoring the replacement prosthesis.

Recent developments in the field have provided devices and methods for mitral valve replacement with reduced invasion and risk to the patient. Such devices typically include a prosthetic valve disposed within the native valve annulus and held in place with an anchor seated against an exterior surface of the heart near the apex, and such anchors must be at least a certain size to seat against the heart with adequate security. Methods of implanting such devices therefore typically require providing an intercostal puncture of significant size to accommodate the anchor. Trauma to the patient increases as a function of the diameter of the puncture. Accordingly, methods and devices for anchoring a prosthetic heart valve that reduce the diameter of any intercostal puncture, or avoid the need for an intercostal puncture altogether, would improve patient outcomes.

### BRIEF SUMMARY OF THE DISCLOSURE

A prosthetic heart valve system is provided in the present disclosure. The prosthetic heart valve system includes a collapsible and expandable stent, a plurality of prosthetic leaflets coupled to the stent, a collapsible anchor, and a tether configured to couple the stent to the anchor, wherein the collapsible anchor includes a flexible frame body defining a plurality of collapsible cells, each cell having at least one side shared with a neighboring cell, a center adjoining point defined by the cells formed in the flexible frame body, the center adjoining point configured to receive the tether, and a first tip end and a second tip end defining a major axis of the flexible frame body, the major axis passing through the center adjoining point.

In some arrangements, the major axis separates a first portion and a second portion of the flexible frame body, the first portion and the second portion being geometrically identical. At least one of the first tip end and the second tip end includes a pointed tip or a plurality of pointed tips. The center adjoining point defines a center opening. The frame body defines a substantially planar surface when in an unbiased condition. At least one of the cells has a curved portion on a side of the cell when the frame body is in an unbiased condition. The curved portion projects upward from a planar surface defined by the frame body when the frame body is in the unbiased condition. The curved portion has a height of between about 10 mm and about 20 mm relative to a planar surface defined by the frame body when the frame body is in the unbiased condition.

In some arrangements, the first tip end is blunted. In some examples, the first tip end is sharp and configured to pierce tissue. The plurality of collapsible cells includes a minimum of four diamond-shaped cells, and the frame body has substantially diamond shape. In another embodiment, the anchor further comprises a first leg coupled to a first periphery support of the frame body and a second leg coupled to a second periphery support of the frame body, the center adjoining point being positioned between the first leg and the second leg. The tether includes a first end fixedly coupled to the stent, and a second free end, a middle portion of the tether between the first end and the second end configured to loop through the center adjoining point of the anchor. The anchor includes a fabric layer or mesh layer disposed thereon. The frame body of the anchor is formed as a unitary piece.

Also disclosed is a method of implanting a prosthetic heart valve into a heart of a patient. The method includes disposing a collapsible anchor in a rolled-up configuration within a delivery tube near a distal end of the delivery tube, the anchor including a flexible frame body defining a plurality of collapsible cells and a center adjoining point defined by the cells formed in the flexible frame body, advancing the delivery tube into a ventricle of the heart of the patient, after the delivery tube is positioned within the ventricle, forming a puncture in a ventricular wall of the heart of the patient, advancing the distal end of the delivery tube through the puncture in the ventricular wall to locate the distal end of the delivery tube through the ventricular wall of the heart, deploying the anchor from the delivery tube to locate the anchor outside the heart wall in an expanded configuration, implanting the prosthetic heart valve into the heart, and coupling a tether to one or both of the anchor and the prosthetic heart valve to anchor the prosthetic heart valve in the heart.

In some arrangements, the anchor includes a curved surface in the expand configuration, the curved surface of the anchor contacting a correspondingly curved surface of the heart after deployment. The tether includes a first end fixedly coupled to the anchor while the anchor is disposed in the delivery tube.

In some arrangements, the implanting of the prosthetic heart valve into the heart includes advancing the prosthetic heart valve over the tether after the anchor is deployed, and the tether is fixed to the prosthetic heart valve after the prosthetic heart valve is implanted into the heart. The tether includes a first end fixedly coupled to the prosthetic heart valve and a second end looped through the anchor while the anchor is disposed within the delivery tube, and the second end of the tether is pulled to tension the tether after the anchor is deployed and after the prosthetic heart valve is implanted into the heart.

### BRIEF DESCRIPTION OF THE DRAWINGS

FIG. 1 is an exploded view of an exemplary prosthetic cardiovascular valve.

FIG. 2 is an opened and flattened view of an unexpanded inner frame of the prosthetic valve of FIG. 1.

FIGS. 3 and 4 are side and bottom views, respectively, of the inner frame of FIG. 2 in an expanded configuration.

FIG. 5 is an opened and flattened view of an unexpanded outer frame of the prosthetic valve of FIG. 1.

FIGS. 6 and 7 are side and top views, respectively, of the outer frame of FIG. 5 in an expanded configuration.

FIGS. 8-10 are side, front, and top views, respectively, of an assembly of the inner frame of FIGS. 2-4 and the outer frame of FIGS. 5-7, all in an expanded configuration.

FIG. 11A is a perspective view of an example of an anchor for the prosthetic valve according to aspects of the disclosure.

FIG. 11B is a perspective view of another example of an anchor for the prosthetic valve according to aspects of the disclosure.

FIG. 12A is a perspective view of another example of an anchor for the prosthetic valve according to aspects of the disclosure.

FIG. 12B is a side view of the anchor of FIG. 12A along the cut-along line A-A′ according to aspects of the disclosure.

FIG. 12C and 12D are different examples of an end structure of the anchor of FIG. 12A according to aspects of the disclosure.

FIG. 13A is a perspective view of an example of an anchor with a tether attached thereto according to aspects of the disclosure.

FIG. 13B is a side view of an example of an anchor with a tether attached thereto according to aspects of the disclosure.

FIG. 14A is a top view of another example of an anchor according to aspects of the disclosure.

FIG. 14B is another example of the anchor of FIG. 14A according to aspects of the disclosure.

FIGS. 15A-15D illustrate different progressive stages of a transseptal insertion process to implant an anchor in a heart according to aspects of the disclosure.

FIG. 16 illustrates an example utilizing dual anchors in a heart according to aspects of the disclosure.

FIG. 17 illustrates the valve of FIG. 1 implanted in a heart utilizing the anchor of FIG. 13.

FIG. 18A and 18B illustrate a delivery tube in a retracted condition and an extended condition to deliver an anchor for implementation according to aspects of the disclosure.

FIG. 19 illustrates a bottom view around an apex of a heart with the anchor implemented thereon according to aspects of the disclosure.

FIG. 20 is a perspective view of an example of an anchor for the prosthetic valve according to aspects of the disclosure

### DETAILED DESCRIPTION

As used herein, the term “proximal,” when used in connection with a delivery device or components of a delivery device, refers to the end of the device closer to the user of the device when the device is being used as intended. On the other hand, the term “distal,” when used in connection with a delivery device or components of a delivery device, refers to the end of the device farther away from the user when the device is being used as intended. As used herein, the terms “substantially,” “generally,” “approximately,” and “about” are intended to mean that slight deviations from absolute are included within the scope of the term so modified.

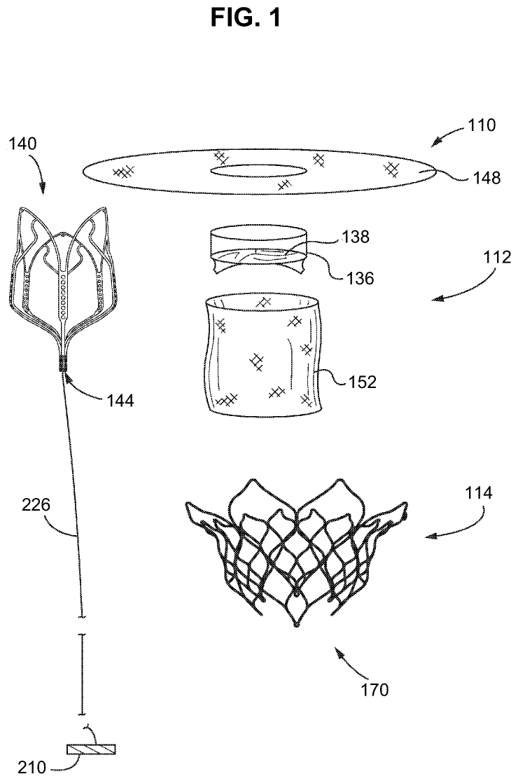

An exemplary prosthetic heart valve 110 as may be used with various embodiments of the present disclosure is shown in an exploded view in FIG. 1. Valve 110 includes an inner structure or assembly 112 and an outer structure or assembly 114. Valve 110 may be coupled to a tether 226 and a collapsible tether anchor 210.

Inner assembly 112 includes an inner frame 140, outer cylindrical wrap 152, and leaflet structure 136 (including articulating leaflets 138 that define a valve function). Leaflet structure 136 may be sewn to inner frame 140 and may use parts of inner frame 140 for this purpose. Inner assembly 112 is disposed and secured within outer assembly 114, as described in more detail below.

Outer assembly 114 includes outer frame 170. Outer frame 170 may also have in various embodiments an outer frame cover of tissue or fabric (not pictured) or may be left without an outer cover to provide exposed wireframe to facilitate in-growth of tissue. Outer frame 170 may also have an articulating collar or cuff (not pictured) covered by a cover 148 of tissue or fabric.

Tether 226 is connected to valve 110 by inner frame 140. Thus, inner frame 140 includes the tether 226, a connecting or clamping portion 144 by which inner frame 140, and by extension valve 110, is coupled to tether 226.

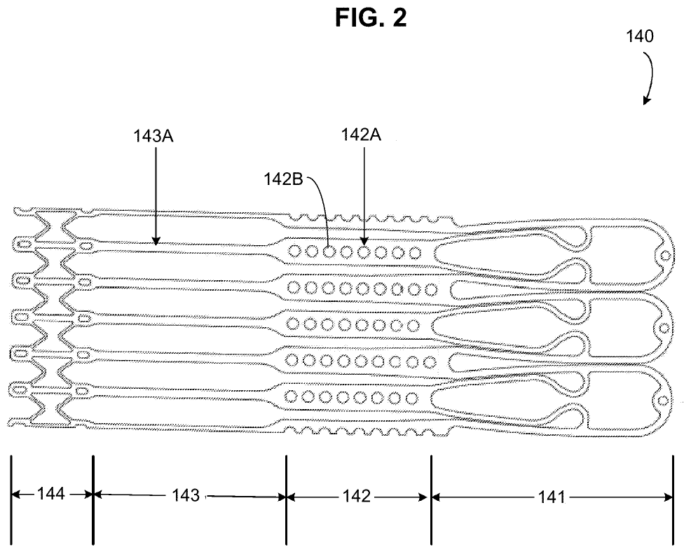

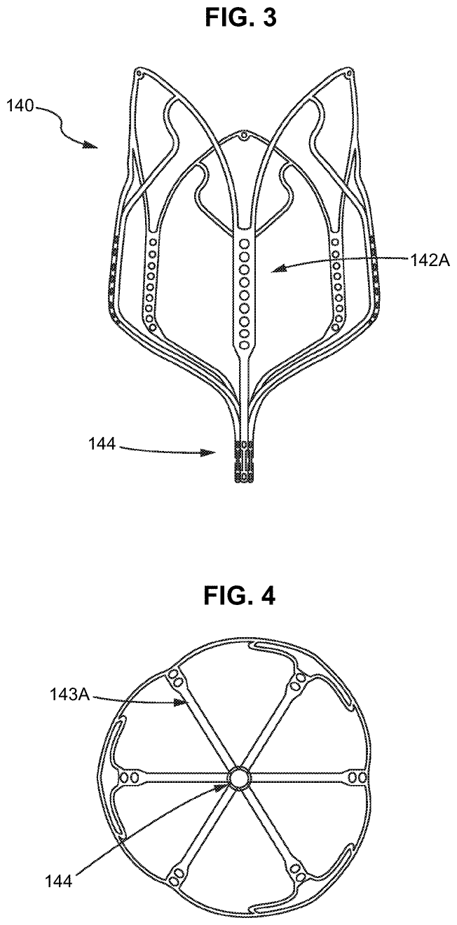

Inner frame 140 is shown in more detail in FIGS. 2-4. Inner frame 140 can be formed from a milled or laser-cut tube of a shape-memory material such as, for example, nitinol. Inner frame 140 is illustrated in FIG. 2 in a compressed or initial state, i.e., as milled or laser-cut, but cut longitudinally and unrolled into a flat sheet for ease of illustration. Inner frame 140 is shown fully expanded, i.e., to the final, deployed configuration, in the side view and bottom view of FIGS. 3 and 4, respectively. Inner frame 140 can be divided into four portions corresponding to functionally different portions of inner frame 140 in final form: apex portion 141, body portion 142, strut portion 143, and tether connecting portion 144. Strut portion 143 includes six struts, such as strut 143A, which connect body portion 142 to connecting portion 144. A greater or lesser number of struts is contemplated herein.

Connecting portion 144 includes longitudinal extensions of the struts, connected circumferentially to one another by pairs of micro-V's. Connecting portion 144 is configured to be radially collapsed by application of a compressive force, which causes the micro-V's to become more deeply V-shaped, with each pair of vertices moving closer together longitudinally and the open ends of the V shapes moving closer together circumferentially. When collapsed, connecting portion 144 can clamp or grip one end of tether 226, either connecting directly onto a tether line or onto an intermediate structure, in turn, firmly fixed to the tether line. The foregoing is merely exemplary and other techniques can be used to connect tether 226 to connecting portion 144.

In contrast to connecting portion 144, apex portion 141 and body portion 142 are configured to be expanded radially. Strut portion 143 forms a longitudinal connection, and radial transition, between the expanded body portion 142 and the compressed connecting portion 144.

Body portion 142 includes six longitudinal posts, such as post 142A, although the body portion may include a greater or lesser number of such posts. The posts can be used to attach leaflet structure 136 to inner frame 140, and/or can be used to attach inner assembly 112 to outer assembly 114, such as by connecting inner frame 140 to outer frame 170. In the illustrated example, posts 142A include apertures 142B through which connecting members (such as suture filaments and/or wires) can be passed to couple the posts to other structures.

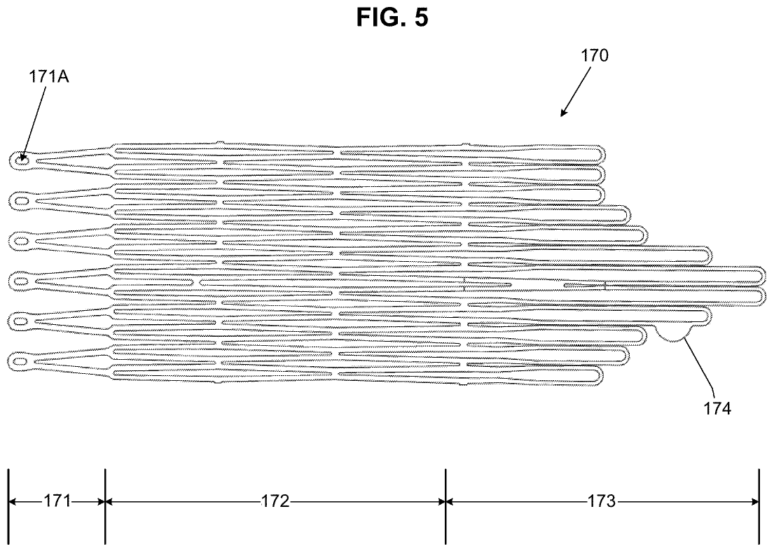

Outer frame 170 of valve 110 is shown in more detail in FIGS. 5-7. Outer frame 170 can be formed from a milled or laser-cut tube of a shape-memory material such as, for example, nitinol. Outer frame 170 is illustrated in FIG. 5 in a collapsed or initial state, i.e., as milled or laser-cut, but cut longitudinally and unrolled into a flat sheet for ease of illustration. Outer frame 170 can be divided into a coupling portion 171, a body portion 172, and a flared portion 173, as shown in FIG. 5. Coupling portion 171 includes multiple openings or apertures 171A by which outer frame 170 can be coupled to inner frame 140, as discussed in more detail below.

Flared portion 173 may include an indicator 174. In one example, indicator 174 is simply a broader portion of the wire frame element of flared portion 173, i.e., indicator 174 is more apparent in radiographic or other imaging modalities than the surrounding wireframe elements of flared portion 173. In other examples, indicator 174 can be any distinguishable feature (e.g., protrusion, notch, etc.) and/or indicia (e.g., lines, markings, tic marks, etc.) that enhance the visibility of the part of flared portion 173 on which it is formed, or to which it is attached. Indicator 174 can facilitate the implantation of the prosthetic valve by providing a reference point or landmark that the operator can use to orient and/or position the valve (or any portion of the valve) with respect to the native valve annulus or other heart structure. For example, during implantation, an operator can identify (e.g., using echocardiography) indicator 174 when the valve 110 is situated in a patient's heart. The operator can therefore determine the location and/or orientation of the valve and make adjustments accordingly.

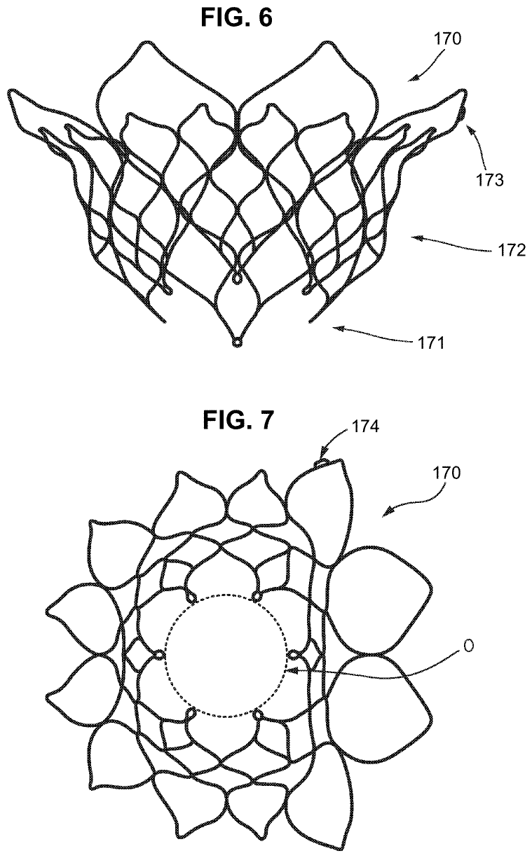

Outer frame 170 is shown fully expanded, i.e., to the final, deployed configuration, in the side view and top view of FIGS. 6 and 7, respectively. As best seen in FIG. 7, the lower end of coupling portion 171 forms a roughly circular opening (identified by “O” in FIG. 7). The diameter of this opening preferably corresponds approximately to the fully deformed diameter of body portion 142 of inner frame 140 to facilitate the coupling together of these two components of valve 110.

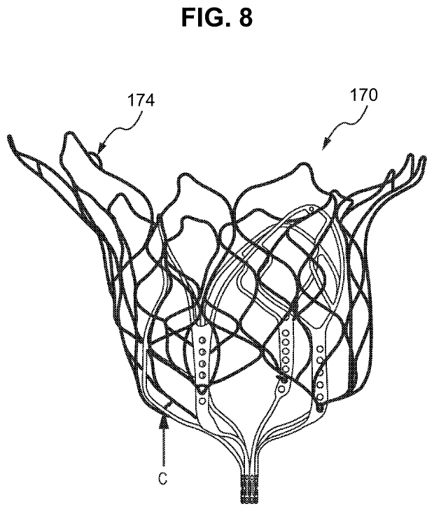

Outer frame 170 and inner frame 140 are shown coupled together in FIGS. 8-10 in front, side, and top views, respectively. The two frames collectively form a structural support for a valve leaflet structure, such as leaflet structure 136 in FIG. 1. The frames support leaflet structure 136 in the desired relationship to the native valve annulus, support the coverings for the two frames to provide a barrier to blood leakage between the atrium and ventricle, and couple to the tether 226 (by the inner frame 140) to aid in holding the prosthetic valve in place in the native valve annulus by the connection of the free end of the tether and tether anchor 210 to the ventricle wall, as described more fully below. The two frames are connected at six coupling points (representative points are identified as “C”). In this embodiment, the coupling of the frames is implemented with a mechanical fastener, such as a short length of wire, passed through an aperture 171A in coupling portion 171 of outer frame 170 and a corresponding aperture 142B in a longitudinal post 142A in body portion 142 of inner frame 140. Inner frame 140 is thus disposed within the outer frame 170 and securely coupled to it.

The prosthetic heart valve 110 shown and described in connection with FIGS. 1-10 may be adapted for a transapical delivery (e.g. through a small incision in the chest and through a small incision in the apex of the left ventricle). However, the prosthetic heart valve 110 may also be suited for other delivery routes, such as fully transseptal delivery routes (including transfemoral and transjugular). For fully transseptal delivery routes, it may be desirable for the inner frame 140 and outer frame 170 to be invertible relative to each other in order to collapse down to a smaller size, which may be helpful to more easily allow the prosthetic heart valve to fit into a catheter that is small enough to pass through the patient's vasculature. Such invertible valves are described in greater detail in U.S. Patent Publication No. 2016/0324635, the disclosure of which is hereby incorporated by reference herein. Further, methods and apparatus for fully transseptal prosthetic mitral valve delivery are described in greater detail in U.S. patent application Ser. No. 17/194,378, filed Mar. 8, 2021 and titled “Apparatus and Methods for Minimally Invasive Transapical Access,” the contents of which are hereby incorporated by reference herein.

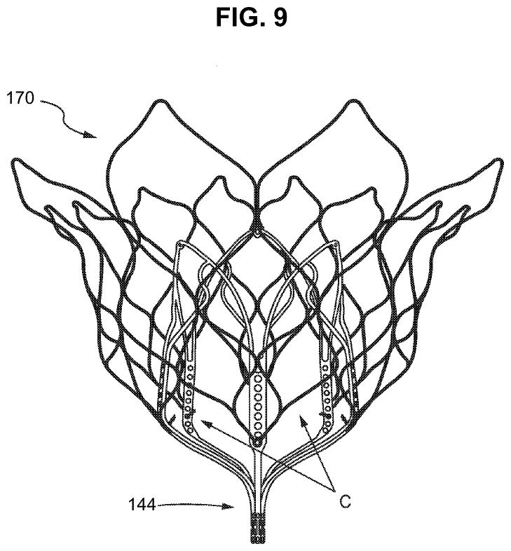

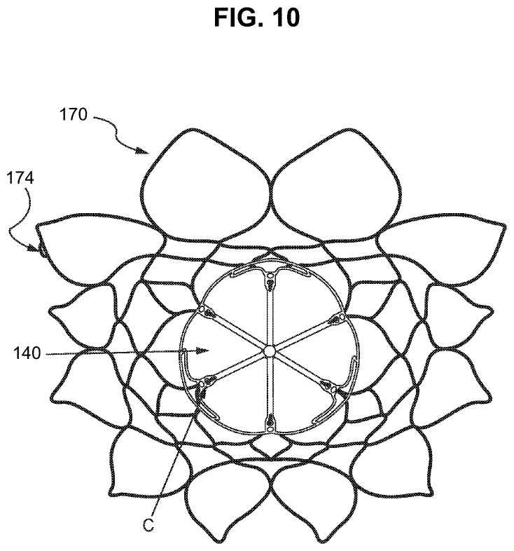

Anchor 210 may take any one of various different forms. For example, if prosthetic heart valve 110 is being delivered transapically, a relatively large opening in the patient's chest is created, and thus the anchor 210 may be relatively large and may not need to be collapsible. However, if prosthetic heart valve 110 (or a similar prosthetic heart valve) is being delivered percutaneously via a transseptal route, for example with an apical puncture being created from within the heart, the anchor 210 may be formed as a collapsible anchor to allow the anchor to collapse to a relatively small size for transcatheter delivery. Various embodiments of collapsible anchors for a prosthetic heart valve are illustrated in FIGS. 11-14. In one example, anchor 210 may be used for a prosthetic mitral heart valve, with the anchor helping to prevent the prosthetic mitral valve from migrating into the left atrium. In other embodiments, anchor 210 may be used for a prosthetic tricuspid valve, with the anchor helping to prevent the prosthetic tricuspid valve from migrating into the right atrium.

In the example depicted in FIG. 11A, the anchor 210 includes a frame 1100. The frame 1100 includes a frame body 1102 comprising a plurality of cells 1104 (shown as 1104a, 1104b, 1104c, 1104d). The frame body 1102 may define substantially a planar surface, such as a flat surface, when in an expanded or unbiased configuration. However, in some embodiments, it may be desirable for the frame body 1102 to have a curved or contoured surface when in an expanded or unbiased configuration. The frame 1100 may be formed from a milled or laser-cut tube of a shape-memory material having elastic and/or memory properties, such as biocompatible metal alloys. Suitable examples of the biocompatible metal alloys include nickel-titanium alloys, such as nitinol, spring stainless steel, tradenamed alloys such as Hastelloy®, aluminum, titanium, nickel, platinum, tantalum, cobalt, chromium, cobalt-chromium alloys, steel-based alloys, such as stainless steel, nickel-based alloys, such as nickel-cobalt-chromium alloys (e.g., tradename Phynox® or Elgiloy®), CoCrMo alloys (e.g., MP35N®), polymers, or combinations or alloys thereof.

The milled or laser-cut tube of the frame 1100 is preferably made of a continuous flexible element which may withstand and spring back from substantial compressive forces imparted thereon during implantation. The flexible nature of the frame 1100 may allow the frame 1100 to be stretched, collapsed and/or compressed as needed during implantation to accommodate different contours, profiles or geometries of hearts from different individual patients so as to provide good anchoring contacts. In one example, the frame 1100 is formed as a unitary piece.

The cell 1104 may be in a quadrilateral form. The cell 1104 may have at least one side shared and/or adjoined with another side from an adjacent or a neighboring cell 1104. For example, a first cell 1104a may have a first side 1105a shared and/or adjoined with a first side 1105b of a second cell 1104b. The first cell 1104a may have a second side 1106a shared and/or adjoined with a first side 1106c from a third cell 1104c. In the example depicted in FIG. 11A, each cell 1104 is configured to have two sides shared and/or adjoined with other two sides from adjacent cells 1104. In other words, frame 1100 may be formed as an integral member with a plurality of struts forming cells, and adjacent cells may have struts in common. In one example, the cell 1104 may be in diamond shape having four sides of substantially equal measures. For example, each side of the cell 1104 may have substantially equal length 1120 between about 3 mm and about 25 mm. Furthermore, inside opposite angles in each cell 1104 may be equal. For example, a first inside angle α₁, β₁ in the first cell 1104a may be substantially equal to a second inside angle α₂, β₂ located horizontally or vertically opposite to the first inside angle α₁, β₁, respectively. In one example, the first inside angle α₁ may be an acute angle substantially equal to the second inside angle α₂ located opposite thereto in a horizontal direction. The first and second inside acute angles α₁, α₂ may have an angle range between about 5 degrees and about 90 degrees. In contrast, a third inside angle β₁ may be an obtuse angle substantially equal to the fourth inside angle β₂ located opposite thereto in a vertical direction. The third and fourth inside angles β₁, β₂ may have a range between about 91 degrees and about 180 degrees. It is noted that the angles formed inside each cell may be altered or adjusted by stretching or compressing the frame body 1102 so as to accommodate different contours, geometries or profiles of hearts from different individual patients so as to provide good anchoring contacts.

It is noted that although the example depicted in FIG. 11A only depicts four cells, it is noted that the cells formed in the frame 1100 may be in any numbers and any suitable shapes. In one example, the plurality of collapsible cells includes a minimum of four diamond-shaped cells. It is preferable, however, that one or more of the cells have a diamond-shape or another shape that is suitable to allow the anchor (or portions thereof) to collapse to a relatively small size to be received within a catheter for transseptal delivery, and then to expand to a relatively large size after the anchor has been delivered to its final position, e.g., across the heart wall to the outer surface of the left or right ventricle.

In one example, the cells 1104 formed in the frame 1100 are adjoined by neighboring sides. A vertex 1190 from each cell 1104 collectively defines a center adjoining point 1150. The center adjoining point 1150 may allow a tether to be tied and/or otherwise coupled thereto. In some examples, the center adjoining point 1150 may be a center aperture 1152, such as a center opening, as shown in FIG. 11B, that allows a tether (such as tether 226) to be looped therethrough as a securing point. It is noted that the center adjoining point 1150 may be in any configuration that allows the tether to be securely coupled thereto as needed.

The frame 1100 may further comprise a major axis 1170 defined by a first tip end 1162a and a second tip end 1162b formed from two adjacent cells 1104b 1104c, as shown in the example of FIG. 11A. The major axis 1170 passes through the center adjoining point 1150. The first and the second tip ends 1162a, 1162b are horizontally aligned along the major axis 1170. A major distance 1160 defined between the two outermost tip ends 1162a, 1162b may be between about 6 mm and about 50 mm. The distance from the center adjoining point 1150 to the first tip end 1162a is substantially the same as or similar to the distance from the center adjoining point 1150 to the second tip end 1162b. The major axis 1170 divides the frame 1100 into a first portion 1180 and a second portion 1182. The first and the second portions 1180, 1182 are symmetrical and geometrically identical. The first and the second portions 1180, 1182 each includes outermost tip ends 1171a, 117 1b aligned in a vertical direction (e.g., in a y direction). Similarly, the distance from the center adjoining point 1150 to the third outermost tip ends 1171a is the same as or similar to the distance from the center adjoining point 1150 to the fourth tip end 1171b. Although illustrated as substantially flat or planar, in the expanded or unbiased condition, frame 1100 may have a curvature in which outermost tips 1171a, 1171b curve or curl toward one another, which may assist in maximizing surface area of contact between the frame 1100 and the heart tissue. This is described in greater detail immediately below.

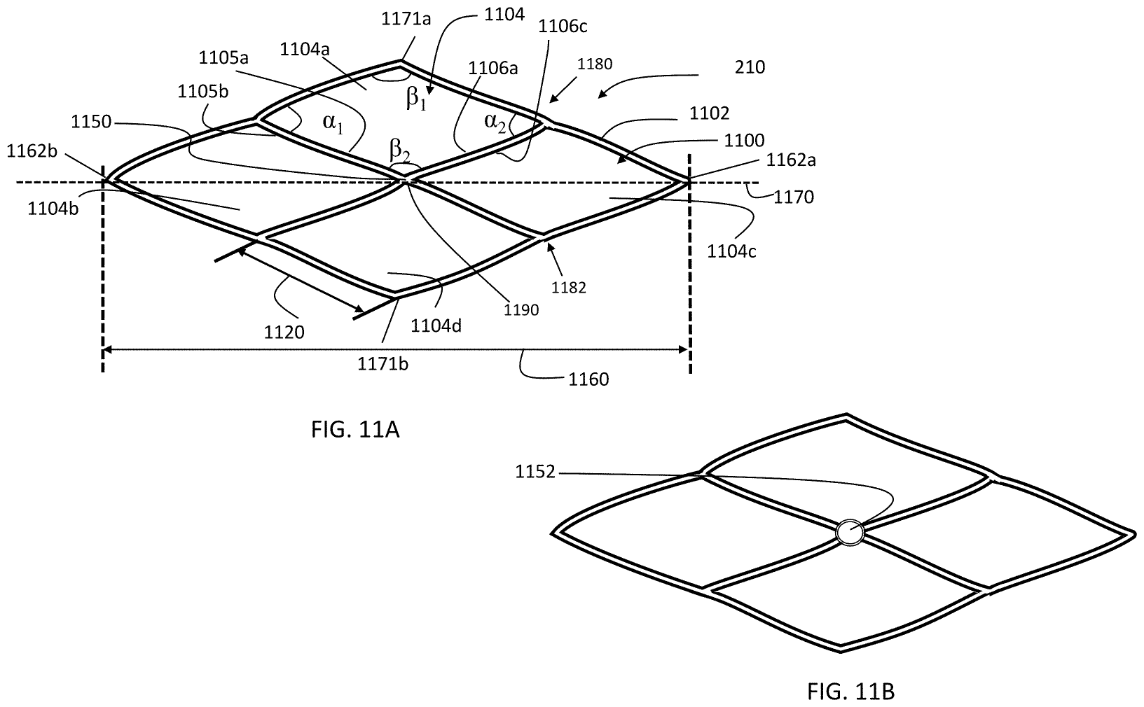

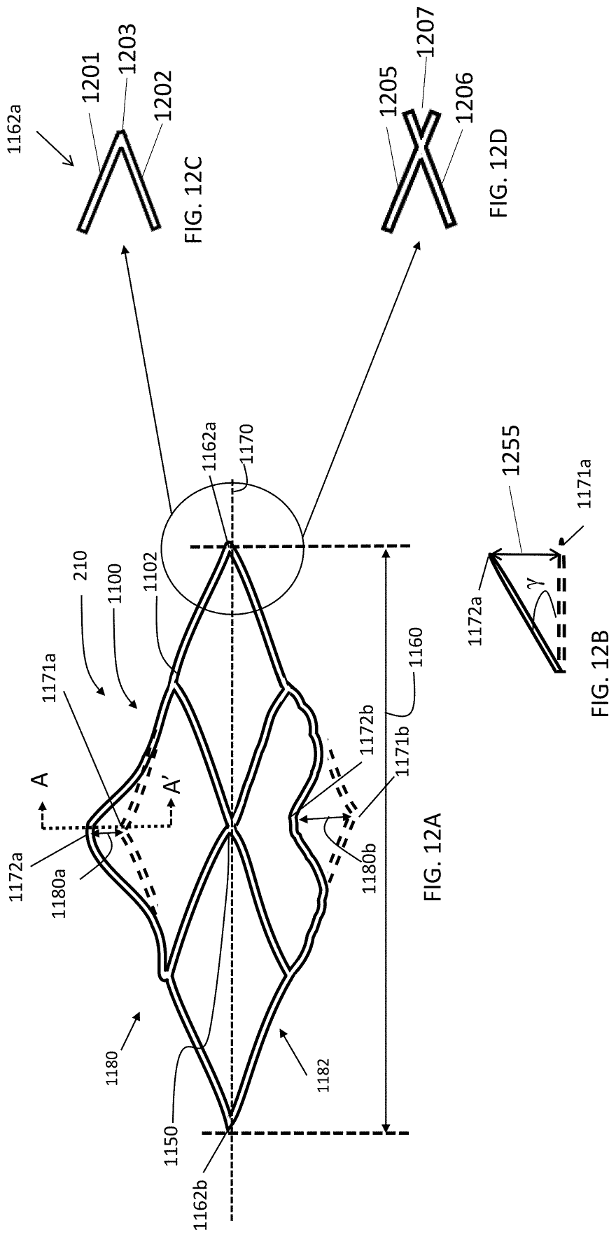

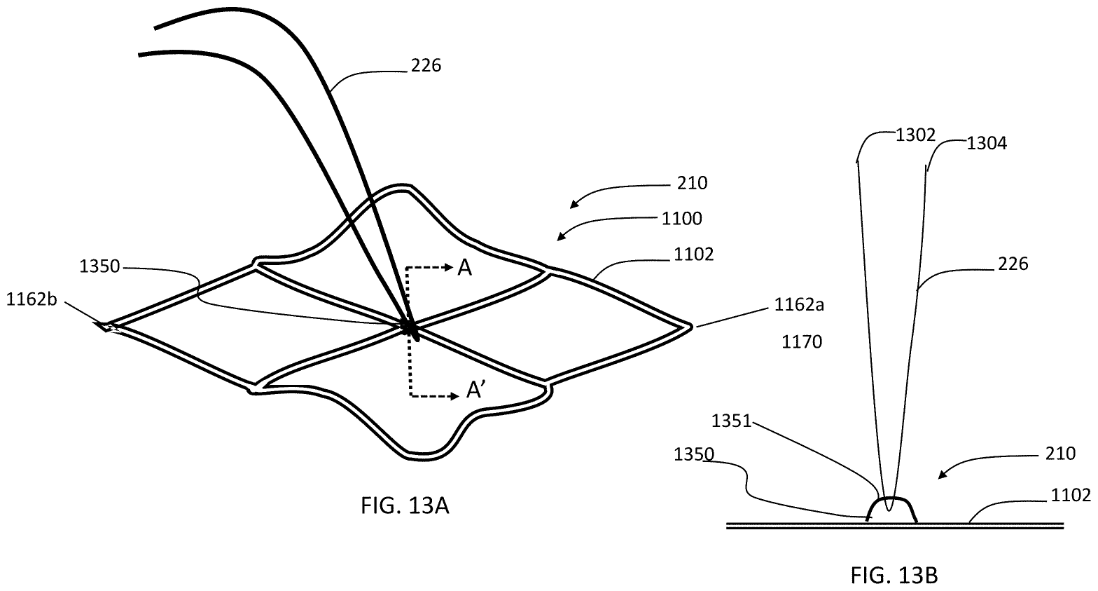

FIG. 12A depicts an embodiment of anchor 210 similar to that of FIG. 11A, but in a partially rolled-up configuration. The third and fourth outermost tip ends 1171a, 1171b aligned in the vertical direction (e.g., in the y direction) may be adjusted or bent to form a curved structure 1172a, 1172b, projecting upward (e.g., in a z direction as shown by the arrow 1180a, 1180b) from the planar surface defined by the frame body 1102. It is believed that the curved structure 1172a, 1172b formed in the anchor 210 may better follow along a contour or an outer curvature of a heart so as to provide a good anchoring and/or contacting surface when implanting the anchor 210 in a position against the tissue of the heart. It is noted that the degree of curvature of the curved structure 1172a, 1172b may vary based on the outer surface profile, curvature, contour or profile of the heart on where the anchor 210 will be placed on. It should be understood that the curvature need not only be limited to the outermost tip ends 1171a, 1172b, but rather additional portions of the frame 1100 may have similar curvature. For example, the entire structure of frame 1100 may have curvature about the major axis 1170.

In the example depicted in FIG. 12A, the curved structures 1172a, 1172b are formed at two sides respectively along the major axis 1170. Similarly, the major axis 1170 passes through the center adjoining point 1150. The first and the second tip ends 1162a, 1162b are horizontally aligned along the major axis 1170. The curved structure 1172a, 1172b are substantially aligned vertically having a substantially equal distance from the curved structure 1172a, 1172b to the center adjoining point 1150 in the major axis 1170.

FIG. 12B depicts a cross sectional view of the curved structure 1172a relative to the third outermost tip end 1171a (prior to bending/curvature) along the cut-along line A-A′. The curved structure 1172a may have an tilt angle y relative to the third outermost tip end 1171a, such as relative to a planar surface defined by the frame body 1102, (prior to curvature adjustment or bending). In one example, the curved structure 1172a may have a height 1255 of between about 10 mm and about 20 mm, such as about 15 mm, relative to a planar surface defined by the frame body 1102 when the frame body is in the unbiased condition. The tilt angle y may be enlarged by bending the curved structures 1172a, 1172b further toward the longitudinal axis 1170 of the frame 1100. When the anchor 210 is configured to be placed in a heart having a relatively acute apex, the tilt angle y is controlled at a relatively large range, such as greater than 30 degrees, to allow the curved structure 1172a, 1172b to better fit the profile of the heart. Although FIG. 12B illustrates the bend of structure 1172a as a substantially straight bend (e.g. a constant angle γ), the bend may be a continuous curvature, for example to form a concave shape intended for contacting the heart tissue.

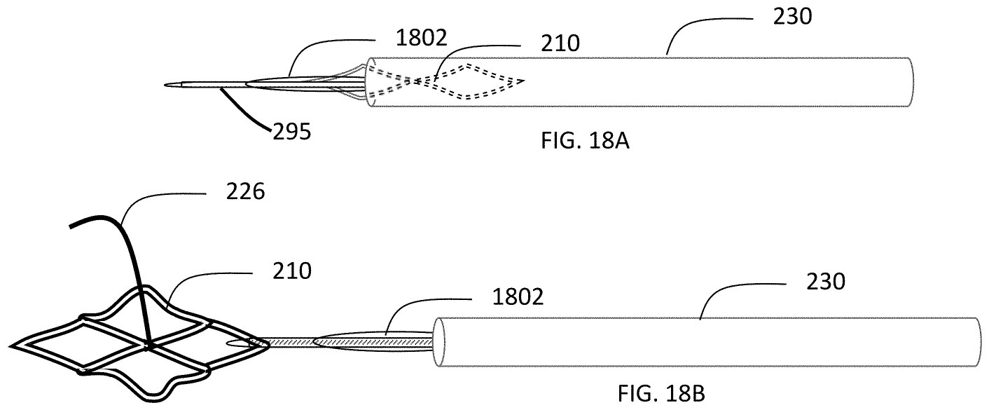

Furthermore, when the anchor 210 is configured to be placed in a delivery device to be delivered for deployment, the curved structures 1172a, 1172b may be further bent to meet or at least partly overlap with each other to roll up the anchor 210 to be placed in a delivery tube from the delivery device. Details regarding how the anchor 210 may be rolled up and placed in a delivery device will be further described below with referenced to FIG. 18A-18B. It should be understood that, however, whether the frame 1100 is completely planar in the expanded unbiased condition or includes some amount of curvature in the expanded unbiased condition, the diamond-shaped cells of the frame allow for the frame to collapse and expand, and the frame may be capable of taking shapes of delivery devices into which the frame is loaded. For example, if the frame 1100 is collapsed and positioned within a tubular catheter, the frame 1100 may lengthen along the major axis 1170, with the frame curling or curving about the major axis to follow the interior curved surface of the catheter in which the frame is positioned.

FIG. 12C and 12D depict two examples of different configurations of the outermost tip ends 1162a, 1162b situated in the major axis 1170. In the example depicted in FIG. 12C, the tip ends 1162a may have the two struts 1201, 1202 join together, forming a tip 1203 with a substantially round, blunt, and/or atraumatic surface. In other words, the two struts 1201, 1202 may be formed as an integral body or piece, forming the tip 1203 with a continuous and curved surface configuration, e.g., a single curved surface. Forming the tip 1203 with a blunted surface may help reduce the likelihood of unintentionally causing trauma to tissue with which the tip comes into contact. In other embodiments, it should be understood that the tip 1203 may be formed with a sharpened point, which may help create a puncture in the heart to assist in passing the anchor from the inside of the heart to the outside of the heart. It should further be understood that the tip ends 1162a, 1162b may be formed differently. In other words, the tip end 1162a that will be the leading end during delivery may be formed to have a sharp tip 1203 because that end may come into contact with tissue first. In that embodiment, it may be desirable to form tip end 1162b to be blunted to avoid unintentional trauma to the tissue. However, in some embodiments it may be preferable to form both tip ends 1162a, 1162b as sharp ends so that it does not matter which end is loaded into the delivery device as the leading end.

In the example depicted in FIG. 12D, the two struts 1205, 1206 may be crossed over at the end, forming a substantially multi-pointed configuration at the tip end 1207. It should be understood that, although the phrase “crossed over” is used, the struts 1205, 1206 and tip end 1207 may be formed from a unitary piece of metal so that there is no material literally “crossing over” other material. Rather, the tip ends 1207 may be thought of as extensions of struts 1205, 1206. This multi-pointed configuration may provide a sharp interface to assist aiding in puncturing through a wall of the heart during implantation, similar to that described above.

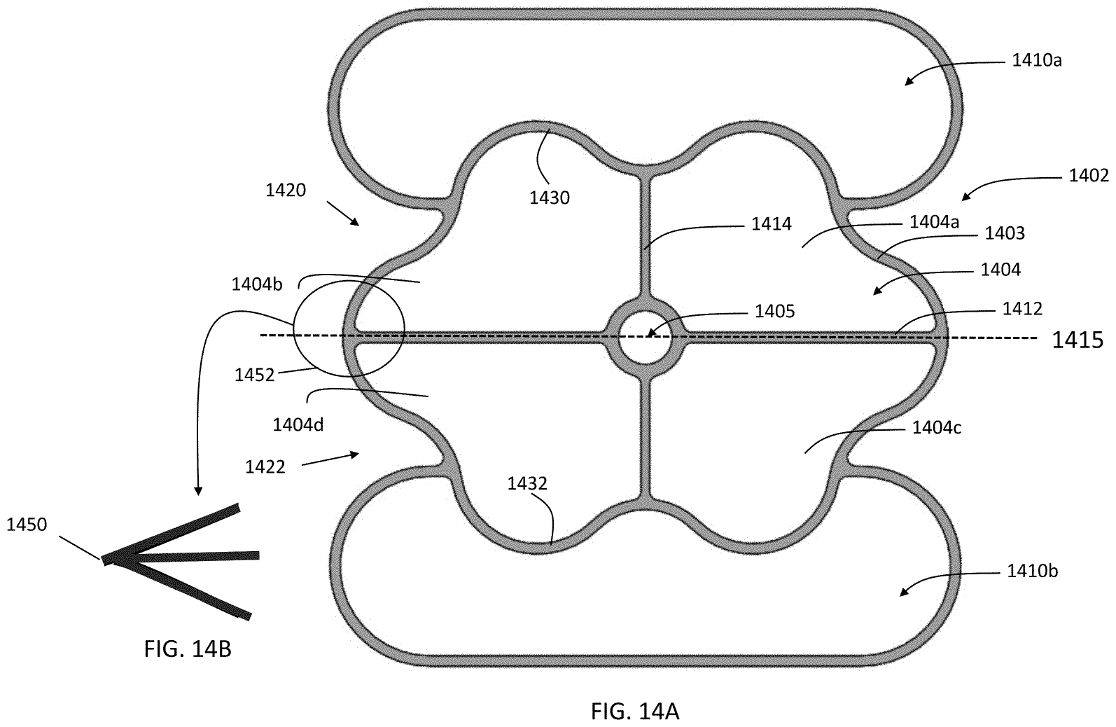

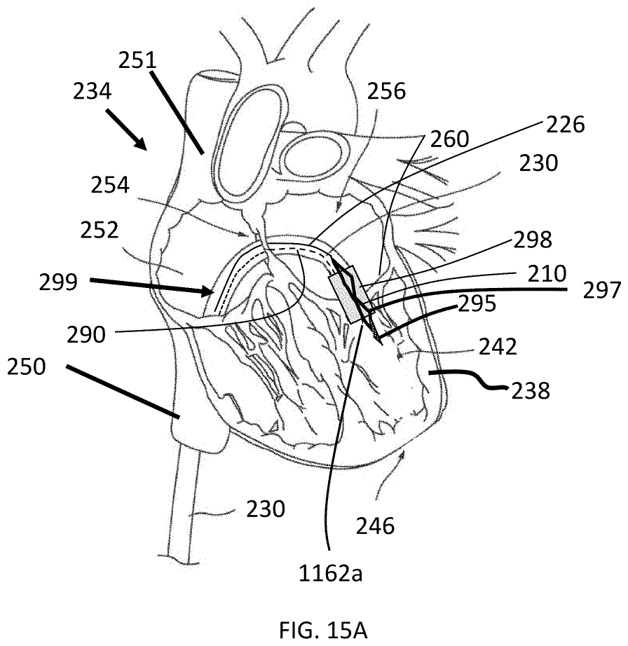

FIG. 13A depicts the anchor 210 with the tether 226 coupled thereto. The tether 226 may be coupled to the anchor 210 in any suitable manner, such as knot tying, mechanical fastening, glue connection, sutures, tabs, and/or other suitable fastening mechanisms. The tether 226 may be an elongated flexible member that may restrict the movement between the heart valve and the anchor 210. In some embodiments, the tether may be a fabric, including a braided fabric, that may provide some amount of elasticity, although in other embodiments the tether may have little or no elasticity. In other embodiments, the tether may be formed of other materials, including metals and metal alloys, such as a thin wire of nitinol. The tethered anchor 210 also inhibits migration of the structures of the heart valve support into the left atrium (for a prosthetic mitral valve) or the right atrium (for a prosthetic tricuspid valve). The tether 226 can be lines, chords, and/or other structures for connecting the heart valve 110 to the anchor 210. In the embodiment depicted in FIG. 13A, the tether 226 includes one long flexible member looping through a center adjoining point 1350, as shown in a cross sectional view along the cut-along line A-A′ as shown in FIG. 13B. It is noted that the center adjoining point 1350 depicted in FIG. 13A-B may include a closed loop hook 1351 that allows the tether 226 to loop therethrough to facilitate attachment of the tether 226 to the anchor 210. The closed loop hook 1351 may be integrated and formed as a unitary part of the frame 1100. It is noted that the closed loop hook 1351 may be formed on either side of the frame 1100. Different features, such as latches, hanging hooks, screws, rings, springs, or the like, may also be utilized to enhance the attachment of the tether 226 to the anchor 210. The tether 226 may have a first end 1302 coupled to the heart valve 110 (for example being fixedly coupled to the tether connection portion 144 by clamping the tether connection portion to the first end 1302) and a second end 1304 that may be pulled through or around the center adjoining point 1150, serving as a pulley device to allow adjustment of tension and/or length between the heart valve 110 and the anchor 210, as described in greater detail below. In some examples, the tether 226 can include one, two, three, or more than four flexible members as needed to facilitate tension adjustment between the heart valve 110 and the anchor 210 as needed. In some embodiments, if the tether 226 is a single wire or single line, one end may be fixedly coupled to the anchor 210 prior to the positioning of the anchor on the exterior surface of the heart, and the other end of the tether may be coupled to the prosthetic heart valve after the prosthetic heart valve is implanted and the tether is tensioned to a desired tension level. Structures and methods that would allow such a feature are described in greater detail in U.S. Provisional Patent Application No. 63/001,637 filed Mar. 30, 2020 and titled “Apparatus and Methods for Valve and Tether Fixation,” the disclosure of which is hereby incorporated by reference herein.

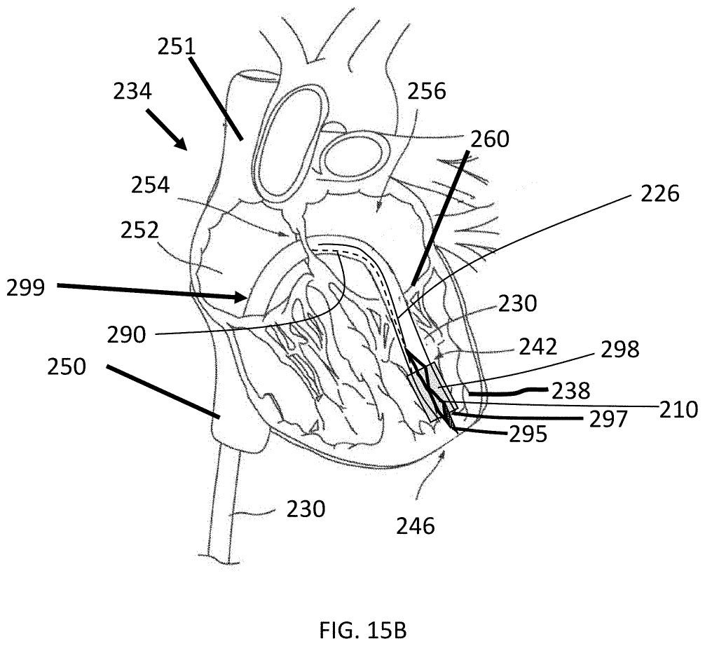

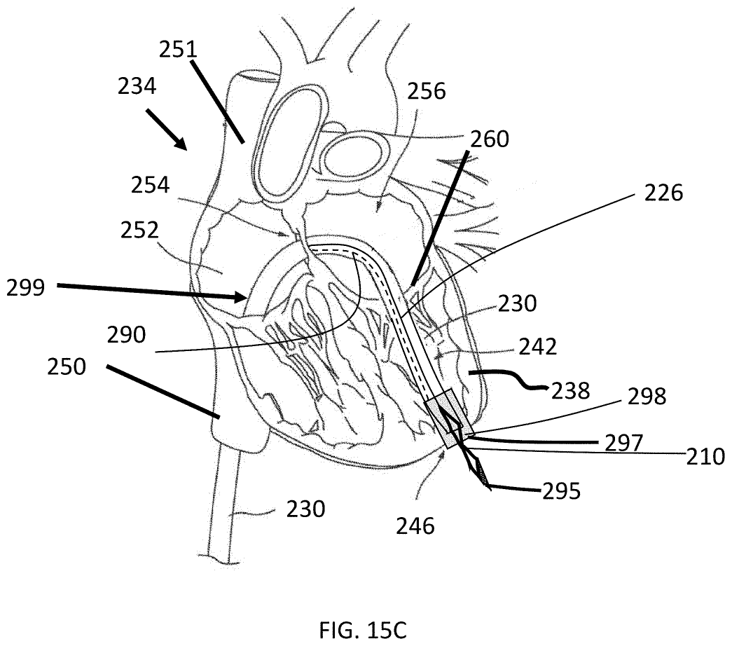

FIG. 14A depicts another example of an anchor 1402 that may be used according to the present disclosure. The anchor 1402 includes a frame body 1403 defining a plurality of cells 1404. The frame body 1403 may define substantially a planar surface, such as a flat surface, when in an unbiased expanded condition. The frame body 1403 may be formed from a milled or laser-cut tube of a shape-memory material having elastic and/or memory properties, such as biocompatible metal alloys. The frame body 1403 may be formed as a unitary piece and may be formed of any of the materials described above in connection with frame body 1102.

The frame body 1403 may be made of a continuous flexible element which may withstand and spring back from substantial compressive forces imparted thereon during implantation. The flexible nature of the frame body 1403 may allow the frame body 1403 to be stretched or compressed as needed during implantation to accommodate different contours, profiles or geometries of hearts from different individual patients so as to provide good anchoring contacts.

In the example depicted in FIG. 14A, a center opening 1405 is defined among the cells 1404 that allows the tether 226 to be looped or pulled therethrough, or which otherwise may facilitate tying the tether 226 to the frame body 1403. A horizontal frame support 1412 and a vertical frame support 1414 may be joined at the center opening 1405. The horizontal frame support 1412 and the vertical frame support 1414 along with a first periphery support 1430 and a second periphery support 1432 in combination define the plurality of cells 1404 in the frame body 1403. The horizontal frame support 1412 also defines a longitudinal axis 1415 that divides a first and a second cell 1404a, 1404b in a first portion 1420 of the anchor 1402 and a third and fourth cell 1404c, 1404d in a second portion 1422 of the anchor 1402. A pair of wings or open cells 1410a, 1410b may each connect to the frame body 1403 in the first portion 1420 and the second portion 1422 respectively. The first leg 1410a is coupled to the first periphery support 1430 along the sides of the first and the second cells 1404a, 1404b. The second leg 1410b is coupled to the second periphery support 1432 along the sides of the third and the fourth cells 1404c, 1404d. It is noted that the first portion 1420 and the second portion 1422 of the frame body 1403 are symmetrical about the longitudinal axis 1415 and are geometrically similar or identical.

During implantation, the tether 226 looped through (or tied to) the center opening 1405 may provide a tension, such as a pulling force, between the anchor 1402 and the heart valve 110 when the tether 226 is pulled by an operator. Accordingly, when the tether 226 is pulled, the contact between the heart tissue and the open cells 1410a, 1410b may provide a counter force, allowing the frame body 1403 close to the center opening to be pulled to a location in close proximity to the outer surface of the heart so as to provide a good anchoring contact for implantation. In other words, when the anchor 1402 is positioned at or near its final desired position on the exterior of the heart, and a pulling force is applied at or near the center opening 1405, the horizontal frame support 1412, the vertical frame support 1414, and the first and second periphery supports 1430, 1432 will all pull toward the heart tissue. However, the open cells 1410a, 1410b are each attached at only two points to peripheries of the corresponding first and second periphery supports 1430, 1432. These connections may significantly reduce the transmission of the tensioning force on the tether 226 to the open cells 1410a, 1410b, which may help ensure that the anchor 1402 maintains its shape and maintains good contact with the tissue of the heart when being tensioned.

In one example, the horizontal frame support 1412 along with the frame body 1403 depicted in FIG. 14A includes round edges that have a smooth, rounded and curved configuration, as indicated by the circle 1452. In another example, a sharp tip end 1450, as shown in FIG. 14B, may be formed where the horizontal frame support 1412 and the frame body 1403 meet, instead of the smooth, rounded and curved configuration shown in FIG. 14A.

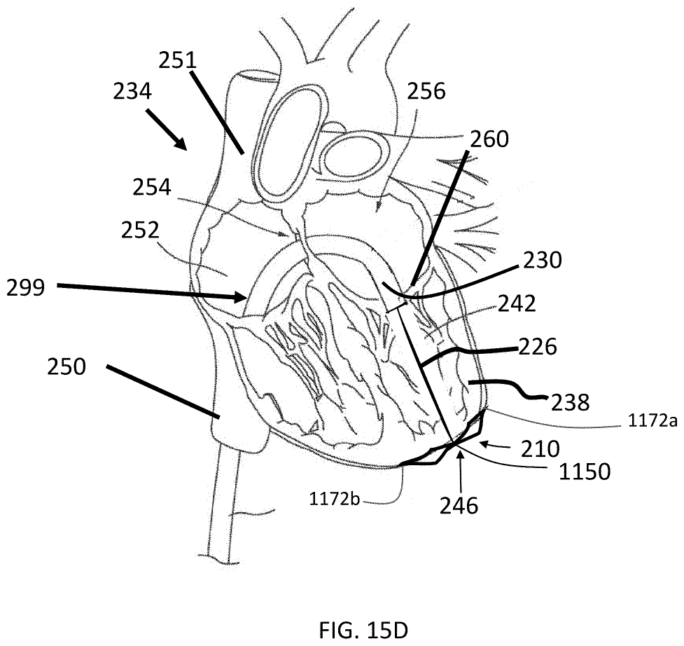

FIG. 15A-15D depicts different stages of deployment of a transseptal insertion process to deliver the anchor 210 to be placed on the heart 234. The illustrated transseptal procedure may include any desirable access route, including a transfemoral access route (via the inferior vena cava 250) or a transjugular access route (via the superior vena cava 251). A steerable catheter or delivery tube 230 is utilized to carry the anchor 210 for delivery. The anchor 210 may be in a rolled-up and/or collapsed configuration to allow the anchor 210 to be inserted and fitted into the delivery tube 230 in a relatively small configuration. An anchor sheath 298 may be positioned distal and integrated with the delivery tube 230. The anchor 210 may be enclosed in the anchor sheath 298 in a collapsed and/or rolled-up configuration and placed inside the delivery tube 230 near or at a distal end thereof. The anchor sheath 298 has a distal opening 297. In some embodiments, for example embodiments in which the anchor 210 has a sharp leading end (e.g. tip end 1162a) that is intended to pierce the ventricular wall, the distal opening 297 of the anchor sheath 298 may be configured to expose the tip end 1162a of the anchor 210. In some embodiments, a dilator and/or introducer 295 may be disposed within the delivery tube 230 and/or within the anchor sheath 298. The anchor 210 is curved and/or wrapped around the introducer 295 configured to pierce the ventricular wall. The introducer 295 may include an inflatable balloon that can be inflated during delivery to provide an atraumatic leading end of the system. When desired, the inflatable balloon of the introducer 295 can be deflated and pulled proximally through the delivery tube 230 to expose an open distal end of the anchor sheath 298 and/or delivery tube 230. Examples of such introducers and dilators, and methods of their use, are described in greater detail in U.S. patent application Ser. No. 17/194,378, filed Mar. 8, 2021 and titled “Apparatus and Methods for Minimally Invasive Transapical Access,” the disclosure of which is hereby incorporated by reference herein. The introducer 295 may provide or include a sharp structure, such as a needle, that may assist puncturing through a ventricular wall 238 of the heart 234 prior to passing the anchor 210 across the ventricular wall 238 of the heart 234. If the tether 226 has a distal end that is fixed to the anchor 210, the proximal end of the tether 226 may be routed proximally through the delivery tube 230, and outside the patient where it is accessible by the surgeon. If the tether 226 is looped around the anchor 210, similar to that shown in FIG. 13B, one end of the tether 226 may be fixed to the prosthetic heart valve 110, and the other end of the tether may extend proximally through the delivery tube 230, and outside the patient where it is accessible by the surgeon. It should be understood that, when the anchor 210 is disposed within the delivery tube 230 and/or anchor sheath 298 in a rolled-up configuration, an open space may be provided radially inward of the rolled-up anchor so that other components (including a dilator/introducer and/or other catheter members) can be passed through the anchor while it is rolled up.

In one example, the delivery tube 230 enters the heart 234 through inferior vena cava 250 (e.g. via the femoral vein), travels through right atrium 252, and punctures the atrial septum 254 to enter left atrium 256. The puncture to the septum 254 may be created in any suitable fashion, including via a sharp leading end of the anchor 210, the dilator/introducer 295, or a separate component (e.g. a BRK transseptal needle). The delivery tube 230 is advanced from left atrium 256 through native mitral valve 260, as shown in FIG. 15A.

The delivery tube 230 is then further advanced to left ventricle 242, and to the ventricular wall 238 in preparation for piercing the ventricular wall 238. If the ventricular wall 238 is to be pierced with the dilator/introducer 295 (or a needle, a balloon or similar device provided therewith), the introducer 295 may be positioned against the interior side of the ventricular wall 238 such that the introducer 295 may puncture the ventricular wall 238, as shown in FIG. 15B. The introducer 295 continues to advance and place the distal end of the delivery tube 230 outside of ventricular wall 238 at or near apex 246, as shown in FIG. 15C. If the anchor 210 is to be used to pierce the ventricular wall 238, the sharp tip of the anchor 210 can be advanced distally to pierce the ventricular wall 238. However, in some embodiments, the sharp distal end of the anchor 210 may be positioned distal to the dilator or balloon so that the balloon need not be deflated prior to piercing the ventricular wall 238. After the ventricular wall 238 is punctured, the distal end of the delivery tube 230 and/or anchor sheath 298 may be advanced along the punched path created in the ventricular wall 238 until the ventricular wall is cleared. At this point, the anchor 210 may be deployed outside of heart 238 from the front opening 297 of the anchor sheath 298, as shown in FIG. 15C. It should be understood that, prior to full deployment, the anchor 210 may be pulled back into the anchor sheath 298 if required. For example, if about 25% or more of the length of the anchor 210 still resides within the anchor sheath 298 and/or the delivery tube 230, the process may be reversed to recapture the anchor 210 within the delivery device 299.

In order to facilitate the deployment, a pusher device 290, such as a pusher rod, may be utilized. The pusher device 290 may be any device that has suitable stiffness in compression. For example, the pusher device 290 may have a distal end that abuts a proximal end of the anchor 210, and a proximal end that extends proximally through the delivery device 299, for example outside the patient's body. In order to deploy the anchor 210, the anchor sheath 298 and/or delivery tube 230 may be retracted proximally, while the operator holds the pusher device 290 static. Thus, as the anchor sheath 298 and/or delivery tube 230 are pulled proximally, the anchor 210 is prevented from also being pulled proximally because the pusher device 290 prevents the anchor 210 from moving proximally. Thus, the delivery tube 230 and/or anchor sheath 298 are pulled or retracted proximally back into the heart while the pusher device 290 forces the anchor 210 to deploy out of the delivery device 299 and into the space adjacent the exterior of the heart. Subsequently, the tether 226 may be pulled proximally to orient the anchor 210 in a direction towards the apex 246 of the heart 234, as shown in FIG. 15D. As the anchor 210 is deployed out of the delivery tube 230, the anchor 210 self-expands back into its unbiased or expanded configuration, such as the configuration depicted in FIG. 11A-14. The expanded configuration of the anchor 210 maximizes the surface area of contact between the anchor 210 and the heart, and also ensures that the anchor 210 cannot pass back into the heart through the ventricular puncture. Upon pulling from the tether 226, a pressure is created to orientate the anchor 210 in a manner that allows the center adjoining point 1150, where the tether 226 is tied to, to seal and block the punched hole in the ventricular wall 238 so as to minimize the area of incision and reduce the likelihood of excessive blood loss. The pressure against the ventricular wall 238 resulting from the tension on the anchor 210 (from the tether 226) allows the anchor 210 to rest in a secured position on the heart 234. The curved structures 1172a, 1172b may assist providing a gripping interface to allow securement of the anchor 210 on the sloped surface around the apex 246 of the heart 234, providing a good mating or anchoring surface between the anchor 210 and the heart 234. Such progressive expansion from within a narrow tube results in the anchor 210 adequately securing the valve 110 to the ventricular wall 238 without requiring an intercostal puncture through the patient's chest.

Although not shown, a guide wire may be used to help guide the delivery device 299 to the desired position, whether using a transfemoral approach, a transjugular approach, or any other approach.

Although not shown, the prosthetic heart valve 110 can be deployed after the anchor 210 is secured to the exterior surface of the heart. If the tether 226 has a distal end fixed to the anchor 210, the proximal end of the tether 226 may extend proximally through the delivery device 299 outside of the patient. Then, the prosthetic heart valve 110 may be positioned in a collapsed condition within delivery device 299 (or another delivery device), and the prosthetic heart valve may be threaded over the proximal end of the tether 226. Then, the prosthetic heart valve 110 may be delivered over the tether 226 using the tether as a rail or a guide, until the prosthetic heart valve 110 reaches the native mitral valve (or tricuspid valve) annulus and is deployed therein. When the positioning of the prosthetic heart valve 110 is confirmed, the tether 226 may be pulled proximally to a desired tension, and then the prosthetic heart valve 110 may be fixed to the tether 226 at the desired tension. Then, any excess length of the tether 226 may be cut and removed from the patient, for example via a cautery tool. Options for this portion of the procedure are described in greater detail in U.S. Provisional Patent Application No. 63/001,593, filed Mar. 30, 2020 and titled “Apparatus and Methods for Minimally Invasive Transapical Access,” the disclosure of which is hereby incorporated by reference herein. In other embodiments, the tether 226 may be looped around the anchor 210, such as that shown and described in connection with FIG. 13B. In that embodiment, the first end 1302 of the tether 226 is fixed to the prosthetic heart valve 110 prior to deployment of the anchor 210. Both the first end 1302 and second end 1304 of the tether 226 may be positioned outside the patient during delivery of the anchor 210. Then, after the anchor 210 is deployed, the prosthetic heart valve 110 may be delivered, and as the prosthetic heart valve 110 (and associated first end 1302 of tether 226) advances toward the native heart valve, the second end 1304 of the tether 226 may be pulled proximally. Once the prosthetic heart valve 110 is deployed into the native annulus, the second end 1304 of the tether 226 can be further pulled to tension the tether 226, with the center adjoining point 1150 acting like a pulley. When the desired tension on the tether 226 is achieved, a portion of the tether 226 between second end 1304 and the anchor 210 may be fixed to the prosthetic heart valve 110 or fixed to some part of the first end 1302 located between the prosthetic heart valve 110 and the anchor 210 to lock the tether 226 at the desired tension.

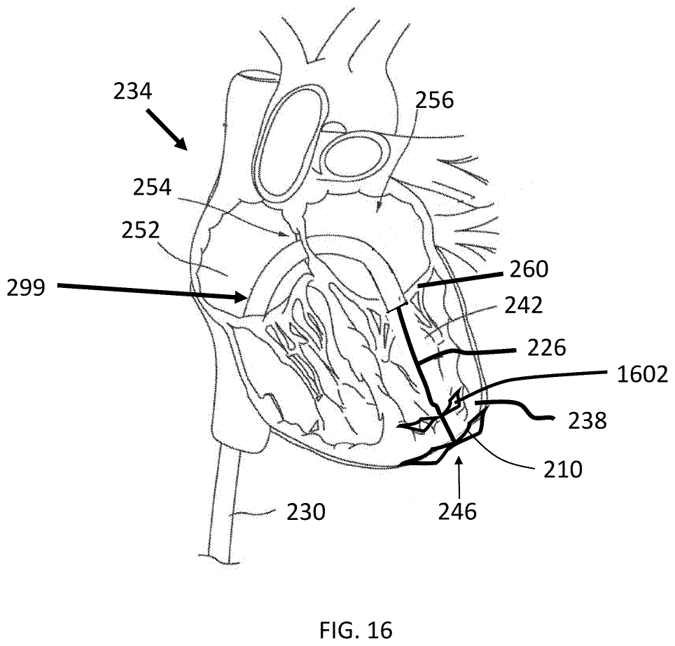

FIG. 16 depicts a secondary anchor 1602 implanted in the heart 234 along with the primary anchor 210. The secondary anchor 1602 may have a similar structure to the primary anchor 210 but with a relatively smaller dimension, such as 20% or 30% smaller of the major distance 1160 compared to the primary anchor 210. The smaller dimension of the secondary anchor 1602 allows the secondary anchor 1602 to rest and sit on an interior surface of the ventricular wall 238 inside the heart 234 in the left ventricle 242. This configuration may assist is further securing the primary anchor 210 in place. Additionally, the primary and the secondary anchors 210, 1602 may, in combination, clamp tissue muscle of the heart 234 where the incision is created so as to block the blood flow therebetween, minimizing the size of the wound and preventing the incision from further tearing off during the following deployment process. However, it should be understood that the secondary anchor is optional and may be omitted. The secondary anchor 1602, if used, may include a central aperture through which the tether 226 is threaded, and the secondary anchor 1602 may be slid distally over the tether 226 and then locked to the tether 226 when it reaches the desired position. Any suitable mechanism can be used for locking the secondary anchor 1602 to the tether 226. For example, the central aperture of the secondary anchor 1602 may include angled hooks that allow the secondary anchor 1602 to be advanced distally over the tether, but the hooks may engage the tether 226 if the secondary anchor 1602 attempts to move proximally relative to the tether 226. However, other locking modalities may be suitable.

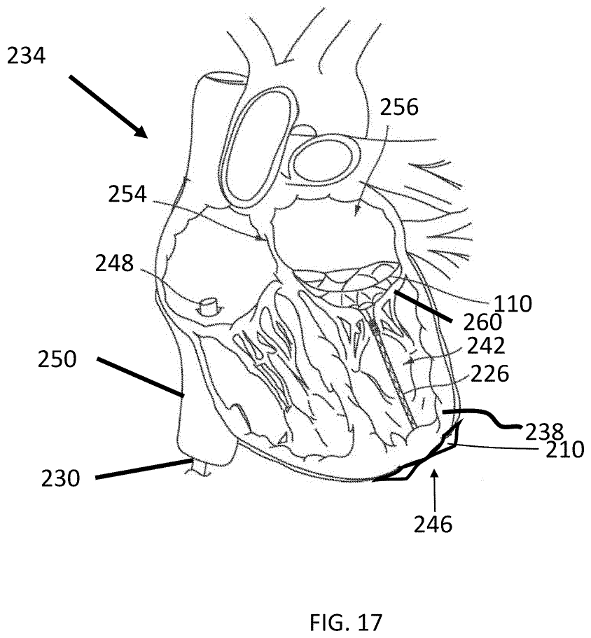

FIG. 17 illustrates the valve 110 implanted in the heart 234 with the anchor 210 seated at or near the apex 246 of heart 234, and the tether 226 locked at the desired tension. The delivery tube 230 has been withdrawn from heart 234, through inferior vena cava 250 in the illustrated example, leaving the valve 110 behind with the anchor 210 connected by the tether 226. It should be understood that the secondary anchor 1602 is not included in the embodiment of FIG. 17.

FIG. 18A-18B depict an example of the delivery tube 230 that may be utilized to deploy the anchor 210. The introducer 295 is placed in the delivery tube 230 to carry the anchor 210 for deployment. As the delivery tube 230 is advanced distally through the vasculature, a balloon dilator 1802 may positioned in the delivery tube 230 at its distal end in an inflated configuration. While the balloon dilator 1802 is inflated, it may provide for a relatively atraumatic leading end of the delivery device 299. The balloon dilator 1802 is inflatable, for example via pushing saline or another material into the balloon for inflation, or pulling saline or another material out of the balloon for deflation. In the example depicted in FIG. 18A, the anchor 210 is in a rolled-up configuration within the delivery tube 230 during delivery, having a hollow center that allows the introducer 295 and the balloon dilator 1802 to be positioned therein and/or therethrough. In some embodiments, the introducer 295 may have a needle or other pointed ember extending therethrough so that, when the delivery device 299 is positioned adjacent the ventricular wall 298, the needle may be advanced to puncture the ventricular wall. In other embodiments, as noted above, a sharp distal end of the anchor 210 may be positioned just distal to the inflated balloon 1802 so that the anchor 210 itself may be used to pierce the ventricular wall. The balloon 1802 may remain inflated as the delivery tube 230 is passed through the punctured ventricular wall, with the inflated balloon helping to dilate the puncture. Prior to deploying the anchor 210, the balloon dilator 1802 may be deflated to allow the anchor 210 to be pushed out of the delivery tube 230 while the open end of the delivery tube 230 is passed through the ventricular wall 238 near the outside apex 246 of the heart 234. The anchor 210 will expand once the anchor 210 is released from the delivery tube 230, as shown in FIG. 18B.

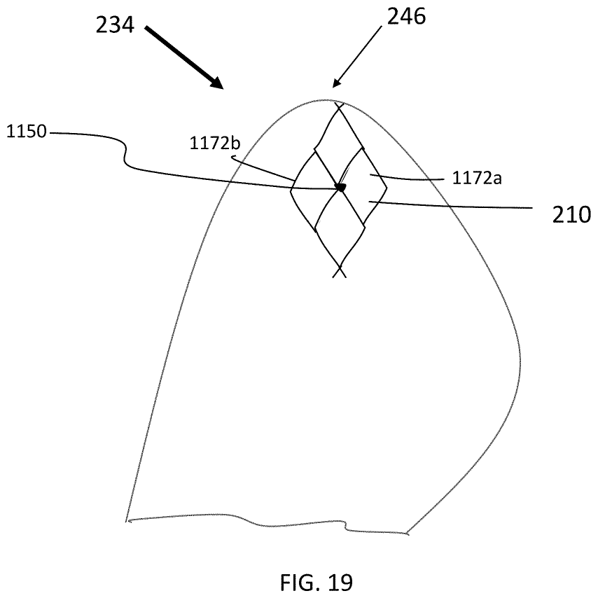

FIG. 19 depicts a bottom view of the heart 234 around the area of the apex 246 where the anchor 210 is placed. After deployment, the anchor 210 is anchored in close contact to the outer surface of the ventricular wall 238 of the heart 234. The curved structures 1172a, 1172b of anchor 210 may help provide a gripping force that allows the anchor 210 to securely adhere and deploy around the apex 246 of the heart 234.

In all of the embodiments of the anchor 210 described above, the anchor is generally shown as a metallic frame. However, it should be understood that additional components may be included. For example, the anchors 210 described above may include one or more layers of fabrics (including synthetic fabrics, such as PET or PTFE), braided meshes (including metal) or the like. The additional layers may help further secure the anchor 210 to the heart, and may also help seal the puncture made in the ventricular wall to minimize any blood loss and to enhance healing of the tissue at the puncture. In some examples, an additional structure or material, such as a collagen, bioabsorbable or fabric plug, may be utilized on the tether. The additional element or material may assist sealing the puncture made in the ventricular wall and enhance healing of the tissue at the puncture.

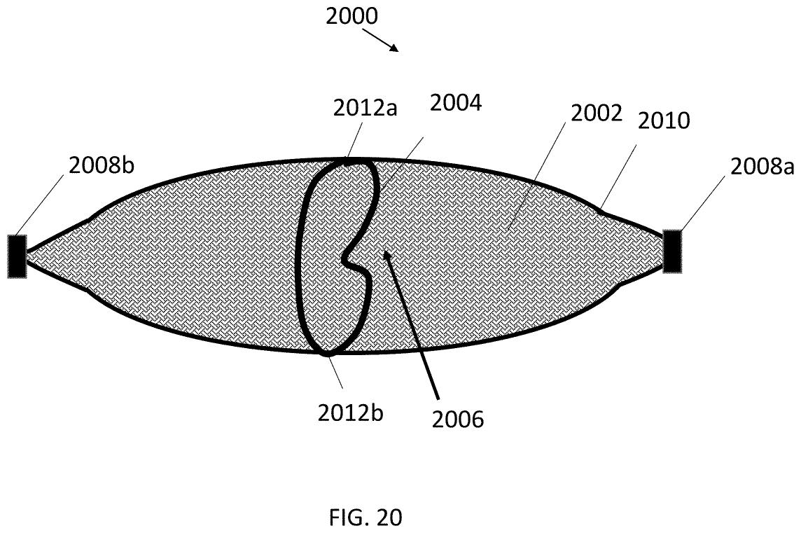

FIG. 20 depicts another example of an anchor 2000 that may be used for deployment according to the present disclosure. The anchor 2000 may include a frame member 2010 and a wire mesh 2002 coupled to the frame member 2010. The frame member 2010 may be formed as a generally diamond-shaped member, such as a unitary piece of nitinol laser cut or nitinol wire formed into a diamond shape. The wire mesh 2002 may be formed from a plurality of strands or wires braided into various three-dimensional shapes and/or geometries. In one example, the wire mesh 2002 is formed by braiding one or more nitinol (or other metal or metal alloys) wires into a mesh. However, the wire mesh 2002 may alternately be formed of any of the materials described above for anchor 210. The frame 2010 may form the periphery or outer circumference of the anchor 2000. Generally, the frame 2010 may provide structural support to allow the anchor 210 to function as an anchor, and may also allow the wire mesh 2002 to take the general shape of the frame 2010. In the example depicted in FIG. 20, a tether securing member 2004 is attached to a center portion 2006 of the wire mesh 2002. The tether securing portion 2004 may take the form of a wire or another structure that is woven through the wire mesh 2002 or otherwise coupled to the wire mesh 2002 and/or the frame member 2010. For example, the tether securing portion 2004 may be fastened on the anchor 2000 by suturing securing points 2012a, 2012b of the tether securing member 2004 to the struts of the frame 2010. The anchor 2000 has two end nodes 2008a, 2008b formed at two lateral ends of the anchor 2000. As the anchor 2000 may be fabricated by a flexible material, the two end nodes 2008a, 2008b may be bent or curved in a manner to accommodate the sloped surface of the outer surface of the ventricular wall 238 around the area of the apex 246 of the heart 234. The degree of curvature may be determined by different geometry or contour of each patience's heart where the anchor 2000 to be deployed. The tether may be tied or otherwise coupled to the tether securing member 2004, including by being looped around the tether securing member 2004 similar to as shown in FIG. 13B. In other embodiments, the tether securing portion 2004 may be omitted and the tether may be directly secured to the wire mesh 2002, for example via tying to the mesh 2002.

As noted above, it may be desirable to include additional fabric material, such as PET or PTFE, in the anchor 2000. In some examples, the wire mesh 2002 may include multiple layers, and one or more layers of fabric material may be positioned between or within the multiple layers of the wire mesh 2002.

Although the anchor(s) described herein are described in conjunction with one particular example of a transcatheter mitral valve, it should be understood that the anchor(s) described herein may be used with any suitable prosthetic heart valve that would benefit from an anchoring system.

To summarize the foregoing, the present disclosure disclosed is a prosthetic heart valve system including an anchor. The prosthetic heart valve system includes a collapsible and expandable stent, a plurality of prosthetic leaflets coupled to the stent, a collapsible anchor, and a tether configured to couple the stent to the anchor, wherein the collapsible anchor includes a flexible frame body defining a plurality of collapsible cells, each cell having at least one side shared with a neighboring cell, a center adjoining point defined by the cells formed in the flexible frame body, the center adjoining point configured to receive the tether, and a first tip end and a second tip end defining a major axis of the flexible frame body, the major axis passing through the center adjoining point.

In some examples, the major axis separates a first portion and a second portion of the flexible frame body, the first portion and the second portion being geometrically identical. At least one of the first tip end and the second tip end includes a plurality of pointed tips. The center adjoining point defines a center opening. The frame body defines a substantially planar surface when in an unbiased condition. At least one of the cells has a curved portion on a side of the cell when the frame body is in an unbiased condition. The curved portion projects upward from a planar surface defined by the frame body when the frame body is in the unbiased condition. The curved portion may have a height of between about 10 mm and about 20 mm relative to a planar surface defined by the frame body when the frame body is in the unbiased condition.

In some examples, the first tip end is blunted. In some examples, the first tip end is sharp and configured to pierce tissue. The plurality of collapsible cells includes a minimum four diamond-shaped cells, and the frame body has substantially diamond shape. The anchor further comprises a first leg coupled to a first periphery support of the frame body and a second leg coupled to a second periphery support of the frame body, the center adjoining point being positioned between the first leg and the second leg. The tether includes a first end fixedly coupled to the stent, and a second free end, a middle portion of the tether between the first end and the second end configured to loop through the center adjoining point of the anchor. The anchor includes a fabric layer or mesh layer disposed thereon. The frame body of the anchor is formed as a unitary piece.

Also disclosed is a method of implanting a prosthetic heart valve into a heart of a patient. The method includes disposing a collapsible anchor in a rolled-up configuration within a delivery tube near a distal end of the delivery tube, the anchor including a flexible frame body defining a plurality of collapsible cells and a center adjoining point defined by the cells formed in the flexible frame body, advancing the delivery tube into a ventricle of the heart of the patient, after the delivery tube is positioned within the ventricle, forming a puncture in a ventricular wall of the heart of the patient, advancing the distal end of the delivery tube through the puncture in the ventricular wall to locate the distal end of the delivery tube through the ventricular wall of the heart, deploying the anchor from the delivery tube to locate the anchor outside the heart wall in an expanded configuration, implanting the prosthetic heart valve into the heart, and coupling a tether to one or both of the anchor and the prosthetic heart valve to anchor the prosthetic heart valve in the heart.

In one example, the anchor includes a curved surface in the expanded configuration, the curved surface of the anchor contacting a correspondingly curved surface of the heart after deployment. The tether includes a first end fixedly coupled to the anchor while the anchor is disposed in the delivery tube.

In one example, the implanting of the prosthetic heart valve into the heart includes advancing the prosthetic heart valve over the tether after the anchor is deployed, and the tether is fixed to the prosthetic heart valve after the prosthetic heart valve is implanted into the heart. The tether includes a first end fixedly coupled to the prosthetic heart valve and a second end looped through the anchor while the anchor is disposed within the delivery tube, and the second end of the tether is pulled to tension the tether after the anchor is deployed and after the prosthetic heart valve is implanted into the heart.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

1. A prosthetic heart valve system comprising:
a collapsible and expandable stent;
a plurality of prosthetic leaflets coupled to the stent;
a collapsible anchor; and
a tether configured to couple the stent to the anchor,
wherein the collapsible anchor includes:
a flexible frame body defining a plurality of collapsible cells, each cell having at least one side shared with a neighboring cell;
a center adjoining point defined by the cells formed in the flexible frame body, the center adjoining point configured to receive the tether; and
a first tip end and a second tip end defining a major axis of the flexible frame body, the major axis passing through the center adjoining point.

2. The system of claim 1, wherein the major axis separates a first portion and a second portion of the flexible frame body, the first portion and the second portion being geometrically identical.

3. The system of claim 1, wherein at least one of the first tip end and the second tip end includes a pointed tip or a plurality of pointed tips.

4. The system of claim 1, wherein the center adjoining point defines a center opening.

5. The system of claim 1, wherein the frame body defines a substantially planar surface when in an unbiased condition.

6. The system of claim 1, wherein at least one of the cells has a curved portion on a side of the cell when the frame body is in an unbiased condition.

7. The system of claim 6, wherein the curved portion projects upward from a planar surface defined by the frame body when the frame body is in the unbiased condition.

8. The system of claim 7, wherein the curved portion has a height of between about 10 mm and about 20 mm relative to a planar surface defined by the frame body when the frame body is in the unbiased condition .

9. The system of claim 1, wherein the first tip end is blunted.

10. The system of claim 1, wherein the first tip end is sharp and configured to pierce tissue.

11. The system of claim 1, wherein the plurality of collapsible cells includes a minimum of four diamond-shaped cells, and the frame body has substantially diamond shape.

12. The system of claim 1, wherein the anchor further comprises a first leg coupled to a first periphery support of the frame body and a second leg coupled to a second periphery support of the frame body, the center adjoining point being positioned between the first leg and the second leg.

13. The system of claim 1, wherein the tether includes a first end fixedly coupled to the stent, and a second free end, a middle portion of the tether between the first end and the second end configured to loop through the center adjoining point of the anchor.

14. The system of claim 1, wherein the anchor includes a fabric layer or mesh layer disposed thereon.

15. The system of claim 1, wherein the frame body of the anchor is formed as a unitary piece.

16. A method of implanting a prosthetic heart valve into a heart of a patient, the method comprising:
disposing a collapsible anchor in a rolled-up configuration within a delivery tube near a distal end of the delivery tube, the anchor including a flexible frame body defining a plurality of collapsible cells and a center adjoining point defined by the cells formed in the flexible frame body;
advancing the delivery tube into a ventricle of the heart of the patient;
after the delivery tube is positioned within the ventricle, forming a puncture in a ventricular wall of the heart of the patient;
advancing the distal end of the delivery tube through the puncture in the ventricular wall to locate the distal end of the delivery tube through the ventricular wall of the heart;
deploying the anchor from the delivery tube to locate the anchor outside the heart wall in an expanded configuration;
implanting the prosthetic heart valve into the heart; and
coupling a tether to one or both of the anchor and the prosthetic heart valve to anchor the prosthetic heart valve in the heart.

17. The method of claim 16, wherein the anchor includes a curved surface in the expand configuration, the curved surface of the anchor contacting a correspondingly curved surface of the heart after deployment.

18. The method of claim 16, wherein the tether includes a first end fixedly coupled to the anchor while the anchor is disposed in the delivery tube.

19. The method of claim 18, wherein implanting the prosthetic heart valve into the heart includes advancing the prosthetic heart valve over the tether after the anchor is deployed, and the tether is fixed to the prosthetic heart valve after the prosthetic heart valve is implanted into the heart.

20. The method of claim 16, wherein the tether includes a first end fixedly coupled to the prosthetic heart valve and a second end looped through the anchor while the anchor is disposed within the delivery tube, and the second end of the tether is pulled to tension the tether after the anchor is deployed and after the prosthetic heart valve is implanted into the heart.
